(19) United States
(12) Patent Application Publication
(10) Pub. No.: US 2006/0063833 A1
(43) Pub. Date: Mar. 23, 2006

(54) Ready-to-use oxaliplatin solutions

(76) Inventors: Edgar Schridde, Hannover (DE); Bernd Merbach, Burgwedel (DE); Stefan-Peter Gimmel, Hannover (DE)

Correspondence Address: KENYON & KENYON LLP, ONE BROADWAY, NEW YORK, NY, 10004, US

(21) Appl. No.: 10/947,604

(22) Filed: Sep. 22, 2004

(51) Int. Cl. A61K 31/28

(52) U.S. Cl. 514/492

(57) ABSTRACT

The invention relates to oxaliplatin solutions that also preferably contain sulfuric acid, phosphoric acid, methane sulfonic acid, ethane sulfonic acid, or para-toluene sulfonic acid. The solutions according to the invention are distinguished by high storage stability.

### FIELD OF THE INVENTION

The invention relates to pharmaceutical preparations of oxaliplatin (I) for parenteral administration. Oxaliplatin (cis-oxalato-(trans-1,2-cyclohexanediamine)-platinum(II); cis-oxalato-(1,2-cyclohexanediamine)-platinum(II), trans-1,2-diaminocyclohexane oxaliplatinum; CAS No. 61825-94-3, Mw 397.3; C₈H₁₄N₂O₄Pt) is a compound first described in 2001 (Pharmeuropa Vol. 13, No. 3, 2001, p. 585-588). It represents a platinum (II) complex having one equivalent trans-1,2-diaminocyclohexane and one equivalent oxalic acid, and has the following chemical structure:

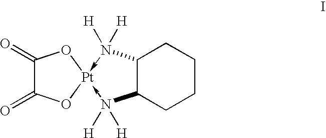

Oxaliplatin is a white, crystalline powder. It is soluble in water, scarcely soluble in methanol, and practically insoluble in ethanol. Oxaliplatin is anti-neoplastic and is used by itself or in combination with 5-fluorouracil and/or folic acid in the therapy of metastatic colorectal cancer. The recommended dose in oxaliplatin therapy is 85 mg/m² body surface area. Like other platinum compounds, oxaliplatin may also be used as a cytostatic in the therapy of a wide variety of cancers such as intestinal cancer, ovarian cancer, or cancer of the upper respiratory tract. Oxaliplatin is administered intravenously to treat a broad range of carcinomas, and thus the agent must be in the form of a solution.

### BACKGROUND OF THE INVENTION

Oxaliplatin solutions known from the related art that are required for parenteral administration are reconstituted directly before being administered to the patient (mammal, preferably human) from a lyophilizate of oxaliplatin, or corresponding crystalline or amorphous solid preparations not manufactured by freeze-drying. However, there are substantial disadvantages associated with the use of such preparations. On the one hand, the procedure to manufacture the lyophilizate is complicated and expensive; on the other hand, reconstitution requires additional steps and represents an undesirable risk for personnel. In particular, the so-called spray-back effect may occur when reconstituting the drug solutions from a dry substance, and this may cause contamination and endanger personnel. Accordingly, any contamination of personnel or inventory by the highly effective cytostatic must be prevented while producing the lyophilizate and reconstituting it. In addition, high demands are placed on the solvents used for reconstitution. They must not be the conventional saline solutions for injection since this would cause the oxaliplatin complex to decompose. Serious difficulties in the oxaliplatin treatment may also arise from errors in handling this lyophilizate such as deviations in the active ingredient concentration, or the microbial contamination of the solution. Given the many potential hazards and errors associated with the use of the lyophilizated agent for producing oxaliplatin solutions, it was therefore desirable to make available ready-to-use pharmaceutical solutions.

Unfortunately, oxaliplatin is not very stable in water, at least at concentrations below 1 mg/ml. In this context and in the rest of the description of the invention, “stability of the solution” is always to be understood as the stability of the oxaliplatin complex in solution, i.e., the long-term relative constancy of the concentration of the initial complex after it dissolves. The insufficient stability of oxaliplatin solutions in water results from the instability of the platinum complex itself whose highly unstable ligands are exchangeable with other, stronger and more reactive nucleophiles, which causes the destruction of the initial complex. The type of additives used in platinum complex solutions is therefore highly crucial.

It is known, for example, that chloride anions cause oxaliplatin to decompose. This is also why oxaliplatin lyophilizates cannot be reconstituted using saline solutions. Even the hydroxide anion from the solvent water is capable of substituting the ligands in a platinum complex. Unstable platinum complexes and hence oxaliplatin must therefore be stabilized in an aqueous solution. Diaquo DACH platinum (II) and diaquo DACH platinum dimer (III) are known examples of decomposition and reaction products of oxaliplatin (oxalato-DACH platinum) brought about by the presence of hydroxide anions.

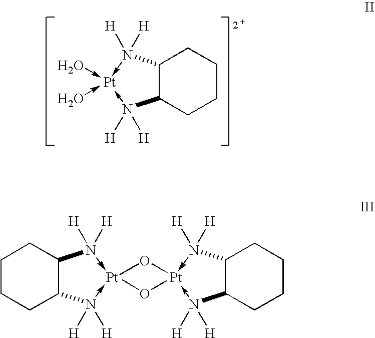

To stabilize the oxaliplatin complexes in aqueous solutions, the concentration or activity of the decomposing anions, such as the hydroxide anion, may be reduced in aqueous solvents. European Patent EP 0 774 963 therefore proposed increasing the concentration of oxaliplatin to at least 1 mg/ml to achieve an acidic pH between 4.5 and 6 and thereby lower the hydroxide anion concentration. However, when the examples from European Patent EP 0 774 963 were tested, only solutions having a pH above 6 were obtained.

The aqueous oxaliplatin solutions described in European Patent EP 0 774 963 are otherwise free of any acid or base, buffer or other additive since their interaction with the oxaliplatin complex and their effect on its stability were unpredictable.

A disadvantage of the approach in European Patent EP 0 774 963 is that the oxaliplatin solutions presented in the patent are only slightly stable. In comparative experiments (see below), it was shown that simple oxaliplatin solutions having compositions from European Patent EP European Patent EP 0 774 963 reveal substantial concentrations of the undesirable degradation products II and III of oxaliplatin after just a few hours. There is therefore a need for aqueous oxaliplatin solutions having greater stability.

As an alternative to simply increasing the concentration, the hydroxide anion concentration may be reduced by adding an acid to stabilize the platinum complex in an aqueous solution. However, there is a danger that the anion resulting from the acid may cause the platinum complex to decompose or change. Beyond stabilizing the agent, is also necessary to slow the formation rate of secondary degradation products by using an acid. Pharmaceutical cis-platinum solutions may be stabilized by adding hydrochloric acid and an additional chloride ion source such as sodium chloride. The corresponding chloride anion does not lead to a breakdown of the cis-platinum complex. As described above, the same additive causes the oxaliplatin complex to degrade undesirably by substituting chloride ions for the unstable ligands.

In addition to adding hydrochloric acid and sodium chloride to an aqueous cis-platinum solution, that is, using the acid of a complex ligand and the complex ligand in free form (corresponding to an isoionic additive) to stabilize the platinum complex, European Patent EP 0 943 331 showed that adding oxalate anions and oxalic acid, i.e. an oxalate buffer, may strongly increase the stability of oxaliplatin solutions in water. Another type of stabilization is used in addition to stabilizing oxaliplatin by lowering the hydroxide anion concentration using the isoionic additive. By adding a ligand of the initial complex as a free species, i.e., a chloride or oxalate anion, the stability of cis-platinum and oxaliplatin is increased.

European Patent EP 0 943 331 also illustrated that neither citric acid, acetic acid, nor the amino acid lysine are suitable for stabilizing the oxaliplatin complex in aqueous solutions. The corresponding anions clearly have a disadvantageous effect on the complex stability of oxaliplatin. In addition, this document describes that the phosphate anion is also incapable of stabilizing aqueous pharmaceutical solutions containing oxaliplatin under neutral conditions. Instead, it was shown that these anions represent unsuitable, highly reactive species that degrade the oxaliplatin complex at the selected pH.

Unfortunately, the type of stabilization of oxaliplatin in aqueous solutions proposed in European Patent EP 0 943 331 is also associated with substantial disadvantages. Neither oxalic acid nor oxalate has been monographed to date in pharmacopeias as a pharmaceutically acceptable adjuvant. Both form water-insoluble salt crystals with calcium and magnesium cations, which are both found in the blood. Particularly in intravenous therapy, higher concentrations of oxalic acid and oxalates may trigger local and systemic side-effects (local injection pain, thrombocyte aggregation, thrombosis, kidneys stones); it is therefore generally undesirable to administer oxalates as components of injectables.

To avoid this disadvantage, it is both necessary and the essential task of the invention to find an adjuvant that is more pharmaceutically suitable and physiologically compatible, and that is equally or more capable of adjusting the pH of the oxaliplatin solution to a physiologically compatible pH that stabilizes the oxaliplatin complex without the accompanying acid anion impairing the stability of the oxaliplatin complex. There are no references to such suitable acids in the related art and science on stabilizing oxaliplatin in aqueous solutions. In particular, there are no references to suitable acid anions.

The object of the invention is therefore to present stable, ready-to-use oxaliplatin solutions that do not have the aforementioned disadvantages of the known solutions. Another object of this invention is to present a method for preparing such solutions.

### SUMMARY OF THE INVENTION

One embodiment of the invention is directed towards a pharmaceutical composition comprising a solution of oxaliplatin in water and an acid, wherein the added acid is not oxalic acid, and wherein the anion of the added acid does not impair the stability of the solution.

In a preferred embodiment, the acid is selected from the group consisting of phosphoric acid, sulfuric acid, methane sulfonic acid, ethane sulfonic acid, para-toluene sulfonic acid, and mixtures thereof.

In a preferred embodiment, the composition further comprises additional tonic, buffering, pH adjusting, solubility improving, or preserving adjuvants.

In a preferred embodiment, the composition has a pH adjusted to a value of from about 3 to about 6. More preferably, the composition has a pH adjusted to a value of from about 3.5 to about 5.0.

In a preferred embodiment, the composition has a oxaliplatin concentration ranging from about 0.025 mg/ml to about 25 mg/ml.

In a preferred embodiment, the composition is not reconstituted from a solid oxaliplatin preparation immediately before being administered to a mammal.

Another aspect of the invention is directed towards a method of treating tumor-related illnesses comprising administering the pharmaceutical composition to a mammal in need thereof.

Another aspect of the invention is directed towards a method of preparing a pharmaceutically acceptable solution of oxaliplatin in water comprising adding one or more organic or inorganic acids, wherein the acid is not oxalic acid, and wherein the anion of the acid does not impair the stability of the solution.

Another aspect of the invention is directed towards a method of preparing a pharmaceutically acceptable solution of oxaliplatin in water comprising adding one or more organic or inorganic acids, wherein the acid is not oxalic acid, and wherein the ratio between oxaliplatin degradation products and oxaliplatin after storage under exclusion of light within 12 months at a temperature of from about 2° C to about 8° C is in the amount of less than about 1 weight percent.

### DETAILED DESCRIPTION OF THE INVENTION

Unless otherwise indicated, the percentages in the present application refer to percent by weight (weight %).

In view of the fact that European Patent EP 0 943 331 discourages the use of phosphate-containing solutions, experiments under the present invention revealed that acids whose corresponding anions are distinguished by low nucleophilia and corresponding buffer systems are capable of stabilizing oxaliplatin in aqueous solution, even at a concentration as low as 0.025 mg/ml.

Suitable acids according to the invention whose anion does not impair the stability of the platinum complex and the solution are in particular the oxoacids of the nonmetals carbon, nitrogen, phosphorus and sulfur; such as carbon dioxide, carbonic acid, nitric acid, nitrous acid, phosphoric acid, ortho-phosphoric acid, phosphorus acid, dihydrogen phosphate salt, acid catena phosphates and their salts, sulfur dioxide, sulfuric acid, sulfurous acid, monohydrogen sulfate salts, alkyl and aryl sulfonic acids, such as methane sulfonic acids, ethane sulfonic acids, and para-toluene sulfonic acid. Other organic acids that may be used are formic acid, lactic acid, ascorbic acid, tartaric acid, succinic acid, benzoic acid and parabenes such as para-hydroxybenzoic acid, para-aminobenzoic acid or ortho-hydroxy benzoic acid. Furthermore, the pH of the oxaliplatin solutions may be adjusted by buffers consisting of the aforementioned acids as well as their water-soluble alkali metal salts and alkaline earth metal salts. Sulfuric acid, phosphoric acid, methane sulfonic acids, ethane sulfonic acids, or para-toluene sulfonic acid are particularly suitable for setting a suitable pH and stabilizing oxaliplatin in an aqueous solution.

In addition to the aforementioned acids, phosphoric acid and sulfuric acid are particularly suitable for adjusting the pH and improving the stability of an oxaliplatin solution, and that their anions are comparatively unreactive with the oxaliplatin complex. Decomposition of the oxaliplatin complex is largely avoided when the pH of an oxaliplatin solution is adjusted using phosphoric acid or sulfuric acid.

The solutions according to the invention include from about 0.025 mg to about 20 mg oxaliplatin per ml water. According to the invention, the solutions are preferably adjusted to a pH of from about 3 to about 6 by adding an acid whose anion does not destabilize the solution.

Particularly preferred acids are phosphoric acid, sulfuric acid, methane sulfonic acids, ethane sulfonic acids, and para-toluene sulfonic acid. A pH-value of from about 3.5 to about 5.0 is particularly preferable.

The oxaliplatin solutions according to the invention may also contain any pharmaceutically acceptable additives in addition to the aforementioned acids or combinations thereof. Examples of these additives are carbohydrates such as lactose, glucose, maltose, fructose, galactose, and/or dextranes.

Adding the aforementioned carbohydrates to the solutions according to the invention allows the concentration of oxaliplatin to be increased without the agent precipitating or crystallizing. Solutions containing carbohydrates are also preferably stabilized by the addition of an acid. It is advantageous to add a carbohydrate as a solubility promoter when the agent concentration is more than about 5 mg/ml. In a preferred embodiment, the solution comprises about 5 mg/ml oxaliplatin, about 55 mg/ml glucose monohydrate, and enough phosphoric acid to adjust the pH to approximately 3.5.

### EXAMPLES

The following examples illustrate the suitability of selected acids (such as phosphoric acid and sulfuric acid) and in particular their anions (phosphate, sulfate) in comparison to oxalic acid (anion: oxalate) as well as preferred embodiments of the invention.

### Example 1

### Comparative Experiments Regarding the Stability of Aqueous Oxaliplatin Solutions Without Additional Additives According to European Patent EP 0 774 963

To determine the stability of unstable oxaliplatin solutions known from European Patent EP 0 774 963, oxaliplatin solutions (C=2.0 mg/ml, with water for injection purposes as the solvent) were stored under exclusion of light at temperatures between 20-25° C., and the relative concentrations of the oxaliplatin degradation products diaquo DACH platinum (II) and diaquo DACH platinum dimer (III) were measured using time-resolved HPLC. The amount of the degradation product oxalic acid is calculated from the amount of platinum-containing degradation products. The results of these investigations are compiled in Table 1.

TABLE 1
Stability of oxaliplatin solutions (C = 2.0 mg/ml) without other additives.
The resulting pH is 6.6; storage temperature: 20-25° C.

| | | Diaquo DACH | |
|---|---|---|---|
| | Diaquo DACH | platinum dimer | |
| | platinum (II)^{a} | (III)^{a} | Oxalic acid^{b} |
| Duration (h) | (% w/w) | (% w/w) | (% w/w) |
| 0 | 0.047 | —^{c} | 0.012 |
| ½ | 0.130 | —^{c} | 0.034 |
| 1 | 0.210 | —^{c} | 0.056 |
| 2 | 0.335 | —^{c} | 0.089 |
| 18 | 0.890 | 0.349 | 0.329 |

^{a}relative to oxaliplatin; amount determined via HPLC.
^{b}relative to oxaliplatin; amount calculated from the amount of degradation products.
^{c}Below the detection limit of 0.03% (w/w).

As may be seen in Table 1, the concentration of the oxaliplatin degradation products increased steadily, and the amount of diaquo DACH platinum (II) exceeded 0.2% after 1 h. After 18 h, a substantial formation of diaquo DACH platinum dimer (III) was noted which is particularly relevant to toxicity. Results summarized in Table 1 indicate that oxaliplatin compositions without stabilizing additives described in European Patent EP European Patent EP 0 774 963 were not sufficiently stable to be stored and pharmaceutically useful over long periods.

Stabilizing additives are therefore required to produce storable oxaliplatin compositions having a pharmacologically acceptable stability over long periods.

### Example 2

### Comparative Experiments Regarding the Stability of Aqueous Oxaliplatin Solutions Stabilized by Adding Oxalic Acid According to European Patent EP 0 943 331, Phosphoric Acid, or Sulfuric Acid

Stability obtained by adding phosphoric acid and sulfuric acid was compared to the stability obtained by adding the corresponding isoionic acid (oxalic acid) known from the related art. European Patent EP 0 943 331 discloses that stable aqueous oxaliplatin solutions (having the known disadvantages) may be obtained by adding oxalic acid.

The influence of the pH and the selected acid on stabilization was investigated using test batches produced at 20-25° C. and stored at 60° C. in which the concentration of oxaliplatin was C=6.6 mg/ml in all the series of experiments, the pH being adjusted using 1 N phosphoric acid and 1 N sulfuric acid in comparison to 1 N oxalic acid. 33 mg oxaliplatin in 5.0 ml solvent having a previously set pH (water for injection purposes having a pH preset using 1 N phosphoric acid, 1 N sulfuric acid or 1 N oxalic acid) was dissolved until clear in a shaker within 16 min. It was then immediately subjected to a HPLC analysis to determine the starting value. After immediately undergoing an initial purity analysis (within 20 min after obtaining a clear solution) the obtained test batches were incubated at 60° C. under exclusion of light and subjected to additional analyses at the provided times (after a total of 6 hours (h) and a total of 7 days (d)). The pH was determined potentiometrically. The results obtained in these experiments are compiled in Tables 2 and 3.

TABLE 2
Stability of aqueous oxaliplatin solutions stabilized by phosphoric acid and
sulfuric acid, and comparison to solutions containing oxalic acid (under accelerated
conditions, T = 60° C.).

| | | | | With phosphoric | | | |
|---|---|---|---|---|---|---|---|
| | | With oxalic acid | | acid | | With sulfuric acid | |
| | | II | III | II | III | II | III |
| pH | Storage | (% w/w) | (% w/w) | (% w/w) | (% w/w) | (% w/w) | (% w/w) |
| 2.0 | Start, 20-25° C. | 0.057 | <0.01 | 0.080 | 0.000 | 0.068 | <0.01 |
| | 6 h, 60° C. | 0.125 | <0.01 | 1.900 | 0.000 | 2.067 | <0.01 |
| | 7 d, 60° C. | 0.135 | <0.01 | 2.36 | <0.01 | 2.540 | <0.01 |
| 2.5 | Start, 20-25° C. | 0.045 | <0.01 | 0.055 | <0.01 | 0.042 | <0.01 |
| | 6 h, 60° C. | 0.106 | <0.01 | 1.110 | <0.01 | 1.117 | <0.01 |
| | 7 d, 60° C. | 0.110 | <0.01 | 1.410 | <0.01 | 1.470 | <0.01 |
| 3.0 | Start, 20-25° C. | 0.016 | <0.01 | 0.068 | <0.01 | 0.025 | <0.01 |
| | 6 h, 60° C. | 0.085 | <0.01 | 0.653 | <0.01 | 0.589 | <0.01 |
| | 7 d, 60° C. | 0.090 | <0.01 | 0.670 | <0.01 | 0.64 | <0.01 |
| 3.5 | Start, 20-25° C. | 0.006 | <0.01 | 0.010 | <0.01 | <0.01 | <0.01 |
| | 6 h, 60° C. | 0.034 | <0.01 | 0.154 | <0.01 | 0.147 | 0.018 |
| | 7 d, 60° C. | 0.090 | <0.01 | 0.260 | 0.010 | 0.220 | 0.010 |
| 4.0 | Start, 20-25° C. | <0.01 | <0.01 | <0.01 | <0.01 | 0.010 | <0.01 |
| | 6 h, 60° C. | 0.062 | 0.066 | 0.082 | 0.040 | 0.088 | 0.040 |
| | 7 d, 60° C. | 0.013 | 0.020 | 0.160 | 0.101 | 0.170 | 0.120 |
| 4.5 | Start, 20-25° C. | 0.007 | <0.01 | 0.010 | <0.01 | 0.011 | <0.01 |
| | 6 h, 60° C. | 0.084 | 0.140 | 0.086 | 0.219 | 0.091 | 0.090 |
| | 7 d, 60° C. | 0.200 | 0.222 | 0.190 | 0.100 | 0.190 | 0.258 |
| 5.0 | Start, 20-25° C. | 0.013 | <0.01 | <0.01 | <0.01 | 0.013 | <0.01 |
| | 6 h, 60° C. | 0.168 | 0.071 | 0.163 | 0.082 | 0.189 | 0.070 |
| | 7 d, 60° C. | 0.190 | 0.090 | 0.190 | 0.090 | 0.180 | 0.090 |
| 5.5 | Start, 20-25° C. | 0.012 | <0.01 | 0.027 | <0.01 | <0.01 | <0.01 |
| | 6 h, 60° C. | 0.182 | 0.096 | 0.171 | 0.074 | 0.180 | 0.090 |
| | 7 d, 60° C. | 0.190 | 0.100 | 0.180 | 0.100 | 0.200 | 0.130 |
| 6.0 | Start, 20-25° C. | <0.01 | <0.01 | 0.022 | <0.01 | 0.021 | <0.01 |
| | 6 h, 60° C. | 0.157 | 0.124 | 0.170 | 0.076 | 0.162 | 0.102 |
| | 7 d, 60° C. | 0.190 | 0.110 | 0.190 | 0.110 | 0.190 | 0.120 |

TABLE 3
Summary of stability measurements under accelerated conditions
(7 d, T = 60° C.; see Table 2).

| | with oxalic acid | | | with phosphoric acid | | | with sulfuric acid | | |
|---|---|---|---|---|---|---|---|---|---|
| pH | A) | B) | C) | A) | B) | C) | A) | B) | C) |
| 2.0 | 72.42 | 0.14 | 0.62 | 4.27 | 2.36 | 3.09 | 4.23 | 2.54 | 2.86 |
| | | <0.01 | | | <0.01 | | | <0.01 | |
| | | 0.48 | | | 0.73 | | | 0.32 | |
| 2.5 | 21.94 | 0.11 | 0.36 | 1.92 | 1.41 | 1.61 | 1.89 | 1.47 | 1.61 |
| | | <0.01 | | | <0.01 | | | <0.01 | |
| | | 0.25 | | | 0.20 | | | 0.14 | |
| 3.0 | 5.73 | 0.09 | 0.15 | 1.21 | 0.67 | 0.69 | 1.02 | 0.64 | 0.65 |
| | | <0.01 | | | <0.01 | | | <0.01 | |
| | | 0.06 | | | 0.02 | | | 0.01 | |
| 3.5 | 1.92 | 0.06 | 0.06 | 0.58 | 0.26 | 0.26 | 0.49 | 0.22 | 0.23 |
| | | <0.01 | | | <0.01 | | | 0.01 | |
| | | <0.01 | | | <0.01 | | | <0.01 | |
| 4.0 | 0.72 | 0.13 | 0.15 | 0.50 | 0.16 | 0.20 | 0.45 | 0.17 | 0.21 |
| | | 0.02 | | | 0.04 | | | 0.04 | |
| | | <0.01 | | | <0.01 | | | <0.01 | |
| 4.5 | 1.01 | 0.02 | 0.34 | 0.77 | 0.19 | 0.29 | 0.66 | 0.19 | 0.28 |
| | | 0.14 | | | 0.09 | | | 0.09 | |
| | | <0.01 | | | <0.01 | | | <0.01 | |
| 5.0 | 0.78 | 0.19 | 0.28 | 0.75 | 0.19 | 0.28 | 0.76 | 0.18 | 0.27 |
| | | 0.09 | | | 0.09 | | | 0.09 | |
| | | <0.01 | | | <0.01 | | | <0.01 | |
| 5.5 | 0.77 | 0.19 | 0.29 | 0.72 | 0.18 | 0.28 | 0.99 | 0.20 | 0.33 |
| | | 0.10 | | | 0.10 | | | 0.13 | |
| | | <0.10 | | | <0.01 | | | <0.01 | |
| 6.0 | 0.78 | 0.19 | 0.30 | 0.75 | 0.19 | 0.30 | 0.86 | 0.19 | 0.31 |
| | | 0.11 | | | 0.11 | | | 0.12 | |
| | | <0.01 | | | <0.01 | | | <0.01 | |

A) % w/w oxalic acid
B) % w/w II; % w/w III; % w/w total of unknown compounds
C) % w/w total of all Pt-containing impurities

The results of the experiments under accelerated conditions (Tables 2 and 3) specify the degradation products and the relationship of the specific impurities at the respective pH. These experiments provide a basis for selecting a narrower pH interval.

The range of optimum stability of oxaliplatin solutions adjusted using phosphoric acid or sulfuric acid and the corresponding stability of the solutions adjusted using oxalic acid is from about pH 3.5 to about 6.0, particularly from about pH 4.0 to about 6.0. The relatively high amount of oxalic acid at an approximate pH of 3 further increases the stability of the solutions adjusted using oxalic acid via product inhibition. However, solutions for parenteral administration having a pH of approximately 3 are undesirable due to the associated injection pain. A higher pH (from about 4 to about 9) is generally tolerated better locally and is therefore generally preferable.

At a pH below 3.0 to 3.5, i.e. in acidic solutions, the amount of undesirable, unknown impurities increased in all the examples. At this pH, no diaquo DACH platinum (II) dimer (III) formation from diaquo DACH platinum (II) (II) was observed. At a pH below pH 3.5 in solutions adjusted using phosphoric acid and sulfuric acid, there was a strong rise in diaquo DACH platinum (II) (II), whereas in solutions adjusted to this pH using oxalic acid, the rise in the concentration of diaquo DACH platinum (II) (II) was suppressed by the much higher amount of free oxalic acid in the solutions. A possible reason is the effective (oxalic acid) product inhibition due to a complex reformation reaction from the degradation products (oxalic acid plus diaquo DACH platinum (II) (II) forming oxaliplatin) according to the mass-action principle of the oxaliplatin complex equilibrium.

A rise in undesirable, unknown impurities is not prevented in solutions adjusted using oxalic acid when the solutions have a large amount of oxalic acid and a strongly acidic pH below pH 3.0.

As a result, the aforementioned kinetics (Table 2 and 3) produced an optimum stability at the following pH intervals:
Solutions adjusted using oxalic acid pH 3.0-6.0
solutions adjusted using phosphoric acid pH 3.5-6.0
solutions adjusted using sulfuric acid pH 3.5-6.0

The experiments illustrate that anions of the acids selected as an alternative to oxalic acid to adjust the pH (phosphoric acid and sulfuric acid, phosphate anions and sulfate anions) did not destabilize the oxaliplatin complex as was the case, by contrast, with citric acid (citrate anions, pH 3, pH 5) and acetic acid (acetate anions, pH 5) in European Patent EP 0 943 331.

### Example 3

### Comparative Experiments Under Real Conditions Regarding the Stability of Aqueous Oxaliplatin Solutions with the Addition of Oxalic Acid, Phosphoric Acid, and Sulfuric Acid in a Limited pH Range

Experimental Design

The test batches were produced at 15-20° C. by dissolving the corresponding amount of oxaliplatin (C=1 mg/ml and C=6.6 mg/ml), water previously adjusted to the target pH (3.5-4.0-5.0) using q.s. 1N oxalic acid, 1N phosphoric acid, and 1N sulfuric acid used for injection purposes as the solvent.

The obtained test batches were sterile filtered after the first purity analyses, aseptically poured into injection vials, incubated protected from light at 2-8° C., and reanalyzed at the above times. The pH was determined by measuring potential at all times.

To compare the degree of stabilization of oxaliplatin solutions according to the invention to other acids beside oxalic acid, the stability obtained by adding phosphoric acid and sulfuric acid was compared to the stability obtained using isoionic additives (oxalic acid) (see Tables 4 and 5). European Patent EP 0 943 331 disclosed that it is possible to produce stable, aqueous oxaliplatin solutions by adding oxalic acid.

The following stabilities were obtained using oxalic acid in the experiments relating to the present invention.

TABLE 4
Stabilities of oxaliplatin solutions (C = 1 mg/ml) at pH = 3.5, pH = 4 and
pH = 5, the pH being adjusted using 1N oxalic acid.

| | | | Diaquo | Diaquo DACH | |
|---|---|---|---|---|---|
| | | | DACH | platinum dimer | Unknown |
| | | Test cycle | platinum II | III | impurities |
| Example | pH | at 2-8° C. | (% w/w) | (% w/w) | (% w/w) |
| pH 3.5 | 3.48 | Start | 0.14 | <0.06 | 0 |
| | 3.48 | 1 mo. | 0.14 | <0.06 | <0.06 |
| | 3.52 | 3 mo. | 0.13 | <0.06 | <0.06 |
| | 3.50 | 12 mo. | 0.12 | <0.06 | <0.06 |
| pH 4 | 3.89 | Start | 0.13 | <0.06 | 0 |
| | 3.99 | 1 mo. | 0.12 | <0.06 | <0.06 |
| | 4.07 | 3 mo. | 0.13 | <0.06 | <0.06 |
| | 4.00 | 12 mo. | 0.13 | <0.06 | <0.06 |
| pH 5 | 4.93 | Start | 0.19 | 0.15 | 0 |
| | 4.99 | 1 mo. | 0.22 | 0.17 | <0.06 |
| | 5.15 | 3 mo. | 0.27 | 0.19 | 0.07 |
| | 5.13 | 12 mo. | 0.31 | 0.25 | 0.08 |

TABLE 5
Stabilities of oxaliplatin solutions (C = 6.6 mg/ml) at pH = 3.5, pH = 4
and pH = 5, the pH being set using 1N oxalic acid.

| | | | Diaquo | Diaquo DACH | |
|---|---|---|---|---|---|
| | | | DACH | platinum dimer | Unknown |
| | | Test cycle | platinum II | III | impurities |
| Example | pH | at 2-8° C. | (% w/w) | (% w/w) | (% w/w) |
| pH 3.5 | 3.44 | Start | 0.14 | <0.01 | 0 |
| | 3.48 | 1 mo. | 0.14 | <0.01 | <0.01 |
| | 3.54 | 3 mo. | 0.14 | <0.01 | 0.01 |
| | 3.50 | 12 mo. | 0.13 | <0.01 | 0.02 |
| pH 4 | 3.81 | Start | 0.14 | 0.03 | 0 |
| | 3.92 | 1 mo. | 0.13 | <0.01 | 0.01 |
| | 4.02 | 3 mo. | 0.12 | <0.01 | 0.02 |
| | 4.01 | 12 mo. | 0.14 | <0.01 | 0.03 |
| pH 5 | 4.93 | Start | 0.17 | 0.16 | 0 |
| | 4.99 | 1 mo. | 0.21 | 0.15 | 0.04 |
| | 5.15 | 3 mo. | 0.26 | 0.18 | 0.07 |
| | 5.03 | 12 mo. | 0.30 | 0.23 | 0.09 |

Investigations of oxaliplatin solutions according to the invention stabilized by adding phosphoric acid were carried out under identical conditions. The stabilities are illustrated in Tables 6 and 7.

TABLE 6
Stabilities of oxaliplatin solutions (C = 1.0 mg/ml) at pH = 3.5, pH = 4
and pH = 5, the pH being adjusted using 1N phosphoric acid.

| | | | Diaquo | Diaquo DACH | |
|---|---|---|---|---|---|
| | | | DACH | platinum dimer | Unknown |
| | | Test cycle | platinum II | III | impurities |
| Example | pH | at 2-8° C. | (% w/w) | (% w/w) | (% w/w) |
| pH 3.5 | 3.44 | Start | 0.12 | <0.06 | 0 |
| | 3.48 | 1 mo. | 0.13 | <0.06 | <0.06 |
| | 3.47 | 3 mo. | 0.13 | <0.06 | <0.06 |
| | 3.43 | 12 mo. | 0.14 | <0.06 | <0.06 |
| pH 4 | 3.92 | Start | 0.13 | <0.06 | 0 |
| | 3.93 | 1 mo. | 0.12 | <0.06 | <0.06 |
| | 4.03 | 3 mo. | 0.12 | <0.06 | <0.06 |
| | 4.05 | 12 mo. | 0.13 | <0.06 | <0.06 |
| pH 5 | 5.02 | Start | 0.18 | 0.14 | 0 |
| | 4.92 | 1 mo. | 0.21 | 0.18 | <0.06 |
| | 5.12 | 3 mo. | 0.26 | 0.20 | 0.07 |
| | 5.10 | 12 mo. | 0.30 | 0.23 | 0.06 |

TABLE 7
Stabilities of oxaliplatin solutions (C = 6.6 mg/ml) at pH = 3.5, pH = 4
and pH = 5, the pH being adjusted using 1N phosphoric acid.

| | | | Diaquo | Diaquo DACH | |
|---|---|---|---|---|---|
| | | | DACH | platinum dimer | Unknown |
| | | Test cycle | platinum II | III | impurities |
| Example | pH | at 2-8° C. | (% w/w) | (% w/w) | (% w/w) |
| pH 3.5 | 3.42 | Start | 0.14 | <0.01 | 0 |
| | 3.44 | 1 mo. | 0.13 | <0.01 | <0.01 |
| | 3.44 | 3 mo. | 0.13 | <0.01 | 0.01 |
| | 3.46 | 12 mo. | 0.14 | <0.01 | 0.02 |
| pH 4 | 4.08 | Start | 0.14 | 0.01 | 0 |
| | 4.00 | 1 mo. | 0.14 | <0.01 | 0.01 |
| | 4.02 | 3 mo. | 0.13 | <0.01 | 0.02 |
| | 4.08 | 12 mo. | 0.14 | <0.01 | 0.02 |
| pH 5 | 5.02 | Start | 0.17 | 0.14 | 0 |
| | 5.08 | 1 mo. | 0.22 | 0.13 | 0.03 |
| | 5.19 | 3 mo. | 0.25 | 0.17 | 0.07 |
| | 5.05 | 12 mo. | 0.28 | 0.21 | 0.10 |

Oxaliplatin solutions stabilized using sulfuric acid were investigated under identical conditions. In these experiments relating to the invention, the following stabilities were obtained using sulfuric acid (Tables 8 and 9).

TABLE 8
Stabilities of oxaliplatin solutions (C = 1.0 mg/ml) at pH = 3.5, pH = 4
and pH = 5, the pH being adjusted using 1N sulfuric acid.

| | | | Diaquo | Diaquo DACH | |
|---|---|---|---|---|---|
| | | | DACH | platinum dimer | Unknown |
| | | Test cycle | platinum II | III | impurities |
| Example | pH | at 2-8° C. | (% w/w) | (% w/w) | (% w/w) |
| pH 3.5 | 3.56 | Start | 0.14 | <0.06 | 0 |
| | 3.54 | 1 mo. | 0.13 | <0.06 | <0.06 |
| | 3.53 | 3 mo. | 0.13 | <0.06 | <0.06 |
| | 3.52 | 12 mo. | 0.14 | <0.06 | <0.06 |
| pH 4 | 4.08 | Start | 0.14 | <0.06 | 0 |
| | 4.02 | 1 mo. | 0.13 | <0.06 | <0.06 |
| | 4.10 | 3 mo. | 0.12 | <0.06 | <0.06 |
| | 4.11 | 12 mo. | 0.14 | <0.06 | <0.06 |
| pH 5 | 4.96 | Start | 0.19 | 0.13 | 0 |
| | 4.98 | 1 mo. | 0.22 | 0.18 | <0.06 |
| | 5.03 | 3 mo. | 0.25 | 0.19 | 0.07 |
| | 5.11 | 12 mo. | 0.31 | 0.24 | 0.08 |

TABLE 9
Stabilities of oxaliplatin solutions (C = 6.6 mg/ml) at pH = 3.5, pH = 4
and pH = 5, the pH being adjusted using 1N sulfuric acid.

| | | | Diaquo | Diaquo DACH | |
|---|---|---|---|---|---|
| | | | DACH | platinum dimer | Unknown |
| | | Test cycle | platinum II | III | impurities |
| Example | pH | at 2-8° C. | (% w/w) | (% w/w) | (% w/w) |
| pH 3.5 | 3.53 | Start | 0.14 | <0.01 | 0 |
| | 3.54 | 1 mo. | 0.14 | <0.01 | <0.01 |
| | 3.56 | 3 mo. | 0.13 | <0.01 | 0.01 |
| | 3.52 | 12 mo. | 0.13 | <0.01 | 0.02 |
| pH 4 | 4.08 | Start | 0.14 | 0.01 | 0 |
| | 4.01 | 1 mo. | 0.14 | <0.01 | 0.01 |
| | 4.03 | 3 mo. | 0.13 | <0.01 | 0.02 |
| | 4.09 | 12 mo. | 0.15 | <0.01 | 0.02 |
| pH 5 | 5.08 | Start | 0.18 | 0.14 | 0 |
| | 5.01 | 1 mo. | 0.23 | 0.13 | 0.03 |
| | 4.99 | 3 mo. | 0.25 | 0.18 | 0.05 |
| | 5.05 | 12 mo. | 0.29 | 0.20 | 0.08 |

Comparing the solutions according to the invention having added phosphoric acid or sulfuric acid to oxaliplatin solutions without additional acid (Example 1) or having added oxalic acid according to related art shows on the one hand that the pH of the stored, aforementioned solutions remains stable during the stability investigation at 2-8° C. with only slight deviations of ±0.2 pH.

On the other hand, it was surprisingly revealed that oxaliplatin solutions whose pH was set to a target pH of 3.5 to 5.0 using phosphoric acid or sulfuric acid are just as stable as the aforementioned oxaliplatin solutions whose pH was also adjusted to 3.5 to 5.0 using oxalic acid.

At the same time and just as surprisingly, these experiments all reveal a relationship between the pH and the total increase in impurities in comparison to the related art oxaliplatin solutions whose pH was adjusted using oxalic acid. The accelerated stability tests demonstrated that other acids besides the corresponding isoionic acid (oxalic acid) and their anions (oxalate) are suitable to stabilize the oxaliplatin complex in aqueous solutions. Only at a very low pH that is practically unsuitable for parenteral administration do the effects of product inhibition and complex reformation occur in oxalic acid-stabilized solutions as a result of the high oxalic acid concentration. Furthermore, the experiments showed that phosphoric acid and sulfuric acid did not generate reactive anions that destabilize the oxaliplatin complex in aqueous solutions.

In particular, it was shown that oxaliplatin solutions in the related art whose pH was adjusted using oxalic acid from approximately 3.5 to 5.0 are no more stable than the oxaliplatin solutions according to the invention that, for example, were adjusted using phosphoric and sulfuric acid to the same acidic pH. To our great surprise, these experiments demonstrated that the solutions according to the invention are just as stable as those that have been stabilized using oxalic acid without having the aforementioned negative effects.

### Example 4

### Dependence of the Stability on Concentration

Oxaliplatin-containing solutions having concentrations of 0.025, 0.25, 1.0, 5.0 and 6.6 mg/ml were prepared in water. The respective pH of 80% of the water for injection required to produce the solutions and heated to 35-45° C. was set using q.s. IN phosphoric acid to pH=3 (±0.2), and pH=4 (±0.2) and pH=5 (±0.2); the corresponding amount of oxaliplatin was dissolved, and the batch was cooled to room temperature. The batches were filled with water for injection and sterile filtered. Finally, they were aseptically poured into 5 ml injection vials, which were sealed air-tight using injection plugs and flanged caps.

The batches were stored protected from light for 12 months at 2-8° C. the amounts were determined via HPLC, and the pH was determined by measuring potential. The results are summarized in Table 10.

TABLE 10
Relation of the concentration to the stability of oxaliplatin solutions. The
pH was adjusted using q.s. phosphoric acid (12 mo. 2-8° C.).

| Concentration | pH 3 | pH 4 | pH 5 |
|---|---|---|---|
| 0.025 mg/ml | 99.4% | 99.2% | 98.4% |
| 0.25 mg/ml | 99.8% | 99.6% | 98.7% |
| 1.0 mg/ml | 100.3% | 99.3% | 99.0% |
| 5.0 mg/ml | 99.9% | 99.1% | 98.6% |
| 6.6 mg/ml | 99.8% | 99.2% | 98.4% |

As clearly illustrated by Table 10, the solutions according to the invention are stabilized by adding phosphoric acid even at concentrations as low as C=0.025 mg/ml.

### Examples 5-99

### Composition of Exemplary, Ready-to-Use Oxaliplatin Solutions According to the Invention

Table 11 presents examples of preferred embodiments of the oxaliplatin solutions according to the invention.

TABLE 11
Preferred oxaliplatin solutions according to the invention

| | Oxaliplatin | | | Carbohydrate, | |
|---|---|---|---|---|---|
| No | (mg/ml) | pH | Acid | hydroxyl derivative | Buffer base |
| 5. | 0.10 | 3.0 | Phosphoric acid | 10 mg/ml glucose | 5 mg/ml disodium |
| | | | | monohydrate, 100 mg/ml | hydrogen |
| | | | | polyethylene | phosphate |
| | | | | glycol 400 | |
| 6. | 0.25 | 5.8 | Phosphoric acid | 10 mg/ml glucose | 20 mg/ml |
| | | | | monohydrate, 200 mg/ml | disodium |
| | | | | polyethylene | hydrogen |
| | | | | glycol 400 | phosphate |
| 7. | 0.25 | 3.8 | Phosphoric acid | 35 mg/ml glucose | 20 mg/ml |
| | | | | monohydrate, 10 mg/ml | disodium |
| | | | | polyethylene | hydrogen |
| | | | | glycol 400 | phosphate |
| 8. | 0.25 | 4.8 | Phosphoric acid | 35 mg/ml glucose | 20 mg/ml |
| | | | | monohydrate | disodium |
| | | | | | hydrogen |
| | | | | | phosphate |
| 9. | 0.25 | 4.8 | Phosphoric acid | 35 mg/ml glucose | 20 mg/ml |
| | | | | monohydrate | disodium |
| | | | | | hydrogen |
| | | | | | phosphate |
| 10. | 0.25 | 3.8 | Phosphoric acid | 35 mg/ml glucose | 0.2 mg/ml |
| | | | | monohydrate | disodium |
| | | | | | hydrogen |
| | | | | | phosphate |
| 11. | 0.25 | 3.4 | Phosphoric acid | 55 mg/ml glucose | 20 mg/ml sodium |
| | | | | monohydrate | lactate |
| 12. | 0.25 | 5.8 | Phosphoric acid | 75 mg/ml glucose | |
| | | | | monohydrate | |
| 13. | 0.25 | 4.8 | Phosphoric acid | | |
| 14. | 0.25 | 3.8 | Sulfuric acid | 40 mg/ml glucose | 0.15 mg/ml |
| | | | | monohydrate | disodium |
| | | | | | hydrogen |
| | | | | | phosphate |
| 15. | 0.25 | 5.8 | Sulfuric acid | 40 mg/ml glucose | |
| | | | | monohydrate | |
| 16. | 0.25 | 5.8 | Sulfuric acid | | |
| 17. | 0.25 | 4.8 | Sodium hydrogen | 55 mg/ml glucose | 0.25 mg/ml |
| | | | sulfate | monohydrate | disodium |
| | | | | | hydrogen |
| | | | | | phosphate |
| 18. | 0.25 | 4.2 | Sodium hydrogen | 55 mg/ml glucose | 0.10 mg/ml |
| | | | sulfate | monohydrate | sodium lactate |
| 19. | 0.25 | 3.8 | Sodium hydrogen | 55 mg/ml glucose | |
| | | | sulfate | monohydrate | |
| 20. | 0.5 | 3.2 | Sulfuric acid | 40 mg/ml glucose | |
| | | | | monohydrate | |
| 21. | 0.5 | 3.2 | Phosphoric acid | 40 mg/ml glucose | |
| | | | | monohydrate | |
| 22. | 0.5 | 3.2 | Phosphoric acid | | |
| 23. | 1.0 | 3.4 | Lactic acid | 60 mg/ml glucose | 2.0 mg/ml sodium |
| | | | | monohydrate | lactate |
| 24. | 1.0 | 3.4 | Lactic acid | 10 mg/ml glucose | 0.25 mg/ml |
| | | | | monohydrate | disodium |
| | | | | | hydrogen |
| | | | | | phosphate |
| 25. | 1.0 | 3.6 | Phosphoric acid | 60 mg/ml glucose | 0.10 mg/ml |
| | | | | monohydrate | disodium |
| | | | | | hydrogen |
| | | | | | phosphate |
| 26. | 1.0 | 3.4 | Lactic acid | 40 mg/ml glucose | |
| | | | | monohydrate | |
| 27. | 1.0 | 3.6 | Lactic acid | | |
| 28. | 1.0 | 3.6 | Phosphoric acid | | |
| 29. | 1.0 | 3.6 | Sulfuric acid | | |
| 30. | 2.0 | 3.6 | Phosphoric acid | | 0.5 mg/ml |
| | | | | | disodium |
| | | | | | hydrogen |
| | | | | | phosphate |
| 31. | 2.0 | 3.6 | ethane sulfonic | | |
| | | | acid | | |
| 32. | 2.0 | 3.6 | Phosphoric acid | | |
| 33. | 3.0 | 4.0 | Sulfuric acid | 50 mg/ml glucose | |
| | | | | monohydrate | |
| 34. | 3.0 | 3.0 | Sulfuric acid | 50 mg/ml glucose | |
| | | | | monohydrate | |
| 35. | 3.0 | 3.0 | Phosphoric acid | | |
| 36. | 4.0 | 5.0 | Sodium hydrogen | | |
| | | | sulfate | | |
| 37. | 5.0 | 5.9 | Phosphoric acid | | |
| 38. | 5.0 | 4.7 | Phosphoric acid | | 0.5 mg/ml |
| | | | | | disodium |
| | | | | | hydrogen |
| | | | | | phosphate |
| 39. | 5.0 | 3.0 | Phosphoric acid | | |
| 40. | 5.0 | 3.5 | Phosphoric acid | | 5 mg/ml disodium |
| | | | | | hydrogen |
| | | | | | phosphate |
| 41. | 5.0 | 4.0 | Sulfuric acid | 100 mg/ml | |
| | | | | polyethylene glycol | |
| | | | | 600 | |
| 42. | 5.0 | 3.0 | Phosphoric acid | 55 mg/ml glucose | 0.5 mg/ml |
| | | | | monohydrate | disodium |
| | | | | | hydrogen |
| | | | | | phosphate |
| 43. | 5.0 | 3.0 | Sulfuric acid | | |
| 44. | 5.0 | 3.0 | Sulfuric acid | | |
| 45. | 6.0 | 3.5 | Phosphoric acid | 55 mg/ml glucose | 0.5 mg/ml |
| | | | | monohydrate | disodium |
| | | | | | hydrogen |
| | | | | | phosphate |
| 46. | 6.0 | 3.5 | Phosphoric acid | 55 mg/ml glucose | |
| | | | | monohydrate | |
| 47. | 6.0 | 3.5 | Phosphoric acid | | |
| 48. | 6.0 | 3.8 | Phosphoric acid | | |
| 49. | 6.0 | 3.2 | Phosphoric acid | | |
| 50. | 6.0 | 3.0 | Phosphoric acid | | |
| 51. | 6.0 | 4.8 | Phosphoric acid | | |
| 52. | 6.0 | 3.0 | Lactic acid | | 1.0 mg/ml sodium |
| | | | | | lactate |
| 53. | 6.0 | 3.8 | Lactic acid | 5 mg/ml polyethylene | 2.0 mg/ml sodium |
| | | | | glycol 400 | lactate |
| 54. | 6.0 | 3.5 | Lactic acid | | 2.0 mg/ml sodium |
| | | | | | lactate |
| 55. | 6.0 | 3.5 | Lactic acid | 20 mg/ml fructose | 0.1 mg/ml sodium |
| | | | | | lactate |
| 56. | 6.0 | 3.5 | Lactic acid | 20 mg/ml fructose | 1.0 mg/ml sodium |
| | | | | | lactate |
| 57. | 6.0 | 3.3 | Lactic acid | 50 mg/ml fructose | 3.0 mg/ml sodium |
| | | | | | lactate |
| 58. | 6.0 | 3.3 | Lactic acid | 100 mg/ml fructose | 3.0 mg/ml sodium |
| | | | | | lactate |
| 59. | 6.0 | 3.0 | Lactic acid | | 4.0 mg/ml sodium |
| | | | | | lactate |
| 60. | 6.0 | 3.1 | Lactic acid | | 5.0 mg/ml sodium |
| | | | | | lactate |
| 61. | 6.0 | 3.8 | Lactic acid | | |
| 62. | 6.0 | 3.0 | Phosphoric acid | 55 mg/ml glucose | 2.0 mg/ml sodium |
| | | | | monohydrate | lactate |
| 63. | 6.0 | 3.0 | Phosphoric acid | 55 mg/ml glucose | 0.15 mg/ml |
| | | | | monohydrate | disodium |
| | | | | | hydrogen |
| | | | | | phosphate |
| 64. | 6.0 | 3.5 | Phosphoric acid | | 0.15 mg/ml |
| | | | | | disodium |
| | | | | | hydrogen |
| | | | | | phosphate |
| 65. | 6.0 | 3.5 | Phosphoric acid | 55 mg/ml glucose | |
| | | | | monohydrate | |
| 66. | 6.0 | 3.5 | Phosphoric acid | 55 mg/ml glucose | 2.0 mg/ml sodium |
| | | | | monohydrate | lactate |
| 67. | 6.0 | 3.5 | Phosphoric acid | 55 mg/ml glucose | 6.0 mg/ml sodium |
| | | | | monohydrate | lactate |
| 68. | 6.0 | 3.5 | Phosphoric acid | 55 mg/ml glucose | 0.35 mg/ml |
| | | | | monohydrate | disodium |
| | | | | | hydrogen |
| | | | | | phosphate |
| 69. | 6.0 | 4.0 | Phosphoric acid | | |
| 70. | 6.0 | 4.0 | Phosphoric acid | | 4.0 mg/ml sodium |
| | | | | | lactate |
| 71. | 6.0 | 3.5 | Phosphoric acid | 50 mg/ml glucose | 5 mg/ml disodium |
| | | | | monohydrate | hydrogen |
| | | | | | phosphate |
| 72. | 6.0 | 3.0 | Phosphoric acid | 80 mg/ml glucose | 15 mg/ml |
| | | | | monohydrate | disodium |
| | | | | | hydrogen |
| | | | | | phosphate |
| 73. | 6.0 | 3.0 | Phosphoric acid | 80 mg/ml glucose | 1 mg/ml disodium |
| | | | | monohydrate | hydrogen |
| | | | | | phosphate |
| 74. | 6.0 | 4.0 | Phosphoric acid | 80 mg/ml glucose | 15 mg/ml |
| | | | | monohydrate | disodium |
| | | | | | hydrogen |
| | | | | | phosphate |
| 75. | 6.0 | 3.5 | Sulfuric acid | 65 mg/ml glucose | |
| | | | | monohydrate | |
| 76. | 6.0 | 3.5 | Sulfuric acid | 10 mg/ml glucose | 0.25 mg/ml |
| | | | | monohydrate | disodium |
| | | | | | hydrogen |
| | | | | | phosphate |
| 77. | 6.0 | 3.5 | Sulfuric acid | | |
| 78. | 6.0 | 3.5 | Nitric acid | | |
| 79. | 6.0 | 3.5 | Ethane sulfonic | | |
| | | | acid | | |
| 80. | 6.0 | 3.5 | Methanesulfonic | | |
| | | | acid | | |
| 81. | 6.0 | 3.5 | Sulfuric acid | | |
| 82. | 6.0 | 3.5 | Sulfuric acid | 45 mg/ml glucose | |
| | | | | monohydrate | |
| 83. | 6.0 | 5.8 | Sulfuric acid | 35 mg/ml glucose | 0.55 mg/ml |
| | | | | monohydrate | disodium |
| | | | | | hydrogen |
| | | | | | phosphate |
| 84. | 6.0 | 3.0 | Sulfuric acid | 35 mg/ml glucose | 1.5 mg/ml |
| | | | | monohydrate | disodium |
| | | | | | hydrogen |
| | | | | | phosphate |
| 85. | 6.0 | 3.4 | Sulfuric acid | 30 mg/ml glucose | 2.5 mg/ml |
| | | | | monohydrate | disodium |
| | | | | | hydrogen |
| | | | | | phosphate |
| 86. | 6.0 | 3.8 | Sulfuric acid | 50 mg/ml glucose | 0.15 mg/ml |
| | | | | monohydrate | disodium |
| | | | | | hydrogen |
| | | | | | phosphate |
| 87. | 6.0 | 5.8 | Sulfuric acid | 5 mg/ml glucose | |
| | | | | monohydrate | |
| 88. | 6.0 | 3.0 | Sulfuric acid | 35 mg/ml glucose | |
| | | | | monohydrate | |
| 89. | 6.0 | 3.4 | Sulfuric acid | 35 mg/ml glucose | |
| | | | | monohydrate | |
| 90. | 6.0 | 3.8 | Sulfuric acid | 35 mg/ml glucose | |
| | | | | monohydrate | |
| 91. | 6.0 | 3.0 | Sulfuric acid | | |
| 92. | 6.0 | 3.4 | Sulfuric acid | | |
| 93. | 6.0 | 3.8 | Sulfuric acid | | |
| 94. | 7.0 | 3.8 | Phosphoric acid | 100 mg/ml glucose | 0.25 mg/ml |
| | | | | monohydrate | disodium |
| | | | | | hydrogen |
| | | | | | phosphate |
| 95. | 7.0 | 3.4 | Phosphoric acid | 100 mg/ml glucose | |
| | | | | monohydrate | |
| 96. | 7.0 | 3.4 | Phosphoric acid | 200 mg/ml glucose | |
| | | | | monohydrate | |
| 97. | 7.0 | 4.4 | Sulfuric acid | 100 mg/ml glucose | |
| | | | | monohydrate | |
| 98. | 7.0 | 4.6 | Lactic acid | 100 mg/ml glucose | 4.0 mg/ml sodium |
| | | | | monohydrate | lactate |
| 99. | 7.0 | 4.0 | Lactic acid | 100 mg/ml glucose | 1.0 mg/ml sodium |
| | | | | monohydrate | lactate |

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods.

What is claimed is:

1. A pharmaceutical composition comprising a solution of oxaliplatin in water and an acid,
wherein the added acid is not oxalic acid, and
wherein the anion of the added acid does not impair the stability of the solution.

2. The pharmaceutical composition according to claim 1, wherein the acid is selected from the group consisting of phosphoric acid, sulfuric acid, methane sulfonic acid, ethane sulfonic acid, para-toluene sulfonic acid, and mixtures thereof.

3. The pharmaceutical composition according to claim 1, further comprising additional tonic, buffering, pH adjusting, solubility improving, or preserving adjuvants.

4. The pharmaceutical composition according to claim 1, wherein the pH is adjusted to a value of from about 3 to about 6.

5. The pharmaceutical composition according to claim 1, wherein the pH is adjusted to a value of from about 3.5 to about 5.0.

6. The pharmaceutical composition according to claim 1, wherein the oxaliplatin concentration ranges from about 0.025 mg/ml to about 25 mg/ml.

7. The pharmaceutical composition according to claim 1, wherein the composition is not reconstituted from a solid oxaliplatin preparation immediately before being administered to a mammal.

8. A method of treating tumor-related illnesses comprising administering the pharmaceutical composition according to claim 1 to a mammal in need thereof.

9. A method of preparing a pharmaceutically acceptable solution of oxaliplatin in water comprising adding one or more organic or inorganic acids, wherein the acid is not oxalic acid, and wherein the anion of the acid does not impair the stability of the solution.

10. A method of preparing a pharmaceutically acceptable solution of oxaliplatin in water comprising adding one or more organic or inorganic acids, wherein the acid is not oxalic acid, and wherein the ratio between oxaliplatin degradation products and oxaliplatin after storage under exclusion of light within 12 months at a temperature of from about 2° C. to about 8° C. is in the amount of less than about 1 weight percent.
